(19) United States
(12) Patent Application Publication
(10) Pub. No.: US 2007/0161525 A1
(43) Pub. Date: Jul. 12, 2007

(54) SKIN CARE MOISTURIZER COMPOSITION

(76) Inventors: Timothy J. Taylor, Phoenix, AZ (US); Bruce Russell Cox, Scottsdale, AZ (US)

Correspondence Address: SNELL & WILMER, LLP (Dial Corp), ONE ARIZONA CENTER, 400 E. VAN BUREN, PHOENIZ, AZ, 85004-2202, US

(21) Appl. No.: 11/615,296

(22) Filed: Dec. 22, 2006

Related U.S. Application Data
(60) Provisional application No. 60/755,585, filed on Dec. 29, 2005.

(51) Int. Cl. A61K 8/00; A61K 8/18

(52) U.S. Cl. 510/130; 424/70.1

(57) ABSTRACT

The present invention comprises a skin care moisturizing composition that quickly provides relief from the visual appearance of dry skin, particularly the ashen appearance of darker complexioned individuals. The invention further comprises a moisturizing composition that provides intermediate term, long term, and continuous relief of dry skin and its attendant symptoms. The moisturizing composition comprises a protease enzyme that facilitates the digestion of corneodesmosomal linkages between skin corneocyte cells.

### CROSS REFERENCE TO RELATED APPLICATION

This Application claims priority to and the benefit of U.S. Provisional Patent Application No. 60/755,585 which was filed on Dec. 29, 2005 and entitled “Skin Care Moisturizer Composition” wherein such application is incorporated in its entirety by reference.

### FIELD OF INVENTION

This invention relates to personal care compositions. In particular, this invention is directed towards personal care compositions intended for use as conditioners and or moisturizers. More specifically, this invention is intended to provide care and protection to the skin of a user and to ameliorate the ashen look of dry skin.

### BACKGROUND OF THE INVENTION

Personal care compositions intended to protect, moisturize, and otherwise beautify the skin of humans are well known in the art and include lotions, creams, ointments, gels, and semi-solid compositions that typically contain functional ingredients such as occlusive agents, humectants, and the like. These skin care compositions generally include aesthetic ingredients, such as, colors and fragrances, and include skin feel enhancers such as emollients and the like. Such compositions are typically applied to the skin of the user, using sprays, liquids, gels, mousses, bars, emollients, and the like. The compositions may be applied by means commensurate with the respective medium that the composition comprises. For example, pressurized containers may be used for foams and mousses, pumps may be used for liquids and gels, etc. In some instances, the skin care composition may be impregnated into towelletes, sponges, wipes, etc.

Moisturizers which are used to relieve dry skin have enjoyed some technical and commercial success, but evidence in the art indicates that artisans are still searching for highly effective moisturizers that enjoy widespread consumer acceptance. A major complaint among consumers is that moisturizers often do not work quickly to provide relief from the visual appearance of dry skin and simultaneously provide intermediate term, long term, and continuous relief of dry skin and its attendant symptoms and/or effects. Some of these symptoms include a feeling of dryness, a feeling of itchiness, flaky skin, cracking skin, red skin, and stiff skin.

While many moisturizers are able to increase the moisture content of dry skin, moisturizers available in the art do not directly affect corneocytes, which are a significant contributor to the appearance of dry and flaky skin. Corneocytes are present on the skin and are linked to each other with corneodesmosomes. When the corneodesmosomes are broken down, corneocytes fall away from the skin. When fewer corneocytes are present on the skin, the skin appears to be less dry and less flaky.

Corneocytes that are partially lifted from the surface of the skin, but which still remain attached to the skin, scatter visible light. This light scattering leads to the appearance of dry skin and has a significant effect among moisturizer users who have a darker skin color or pigmentation. The skin of users with a darker skin color or pigmentation is more likely to exhibit a flaky, whitish appearance (called ash) due to the light scattering.

A skin care moisturizing composition is needed to address the above concerns, quickly provide relief from the visual appearance of dry skin, and provide intermediate term, long term, and continuous relief of such dry skin and its attendant symptoms. Moreover, a skin care moisturizer is needed to specifically address the ashen appearance of darker complexioned individuals.

### SUMMARY OF THE INVENTION

As set forth in the detailed description, the present invention includes, in various exemplary embodiments, a moisturizer composition that provides immediate term as well as long term relief of both dry skin appearance and clinical markers of dry skin, including skin barrier function and moisture content. In accordance with an exemplary embodiment of the present invention, a moisturizer composition which comprises a protease enzyme is disclosed to provide rapid visual improvement of dry skin. Such a moisturizer is also effective in treating dry skin for individuals having darker skin coloration or pigmentation, including reducing the visual effects of dry skin in such individuals.

Another embodiment of the invention provides a skin care moisturizer that comprises a first phase, where the first phase is a continuous phase, and a second phase, where the second phase is a dispersed phase. The first phase comprises (i) a liquid, for example, water; (ii) a protease enzyme derived from at least one of a papaya and a pineapple; and (iii) at least one conditioning agent that is water soluble. The first phase, according to various embodiments, may also comprise at least one of a humectant, a conditioning polymer, a color, a pH adjuster, a stabilizer, a preservative, and a vitamin. The second phase comprises a component that is not miscible with water. For example, in various embodiments, the second phase comprises at least one of an oil, a lipid, a fatty acid, and an oil-soluble vitamin.

### DETAILED DESCRIPTION

The detailed description herein makes use of various exemplary embodiments to assist in disclosing the present invention. While these exemplary embodiments are described in sufficient detail to enable those skilled in the art to practice the invention, it should be understood that other embodiments may be realized and that modifications of structures, arrangements, applications, proportions, elements, materials, or components used in the practice of the instant invention, in addition to those not specifically recited, can be varied or otherwise particularly adapted to specific environments, manufacturing specifications, design parameters or other operating requirements without departing from the scope of the present invention and are intended to be included in this disclosure. Thus, the detailed description herein is presented for purposes of illustration only and not of limitation.

It should be appreciated by those skilled in the art that compositions of the present invention are effective at solving the problems encountered in currently marketed and prior art moisturizers. The skin moisturizer of the present invention provides immediate term, intermediate term and long term relief from the clinical markers of dry skin and the visual appearance of dry skin. In an exemplary embodiment of the invention, this relief is provided by compositions that are effective at moisturizing and providing enzymatic removal of partially detached corneocytes.

Turning now to various embodiments of the present invention, a skin care moisturizing composition may be embodied in a cream, lotion, gel, semi-solid, liquid, and the like. These moisturizing compositions are applied to the skin of an individual in a variety of methods, for example, by rubbing, spreading, spraying, massaging, or in any combination of application methods. The moisturizing composition generally contains a base composition comprising an emulsion, also known as a dispersion, wherein the emulsion generally comprises a first phase and a second phase. The first phase of the emulsion comprises a liquid, such as water, and the second phase comprises a component that is not miscible and/or not soluble with water. For example, the second phase may comprise an oil and/or lipid composition.

In accordance with an embodiment of the present invention, the first phase of the emulsion is a continuous phase in an O/W emulsion. In a further embodiment, the first phase is a dispersed phase in a W/O emulsion. The first phase comprises water and/or water-soluble skin conditioning agents, such as humectants, conditioning polymers, and the like. The first phase may also comprise adjuvant ingredients, such as colors, fragrances, pH adjusters, stabilizers, preservatives, vitamins, and the like. These adjuvant ingredients serve various functions, for example, to modify and/or enhance the characteristics of the first phase.

In accordance with an exemplary embodiment of the invention, the first phase comprises a protease enzyme, in particular, a protease enzyme that is highly efficient at digesting the corneodesmosomal linkages existing among skin corneocyte cells present at the surface of the skin. Corneocyte cells contribute to the dryness of the skin and contribute to the ashen appearance of dry skin. In various embodiments of the invention, protease enzymes that are efficient at digesting the corneodesmosomal linkages among skin corneocytes cells are employed. Such protease enzymes, according to an exemplary embodiment, comprise those enzymes found in extracts of papaya and pineapple. Extracts of papaya and pineapple are a rich source of protease enzymes. As the protease enzyme digests the corneodesmosomal linkages, the corneocyte cells are removed from the skin. Thus, the dry skin appearance, which results from corneocytes that are only partially lifted from the surface of the skin and are still attached to the surface of the skin, is reduced.

In accordance with an embodiment of the present invention, the second phase of the emulsion is a dispersed phase in an O/W emulsion. In a further embodiment of the invention, the second phase of the emulsion is a continuous phase in a W/O emulsion. The second phase of the emulsion comprises a skin conditioning agent, for example an oil, a fatty acid, a lipid, an oil-soluble vitamin, and the like.

According to an exemplary embodiment of the present invention, the skin care moisturizer composition is prepared according to skills and processes widely known in the art for preparing water-oil (W/O) emulsions and/or oil-water (O/W) emulsions. The composition comprises, for example, a first phase that comprises an aqueous continuous phase. The first phase further comprises water, glycerol, disodium EDTA, preservative, polyquaternium 10, quaternium 18, and pineapple juice extract. The pineapple juice extract is rich in protease enzyme. The composition further comprises a second phase which comprises an oil phase, and which is a dispersed phase. The second phase further comprises at least one of the following materials: petrolatum, stearic acid, palmitic acid, tocopheryl acetate, sorbitan stearate, and ethoxylated sorbitan stearate.

In accordance with a further exemplary embodiment of the invention, the skin care moisturizer composition is applied to the skin of an individual who has dry skin. Because of the unique skin care composition described herein, the skin is instantly moisturized and further remains perceptively moisturized for twenty four (24) hours or longer. The skin maintains this moisturized characteristic, for example, because of the reduction in dry skin flakes. In accordance with another embodiment, continued use of the moisturizer for twenty one (21) days results in skin that is clinically as well as visually moisturized, smooth, supple, and healthy.

In yet another embodiment of the invention, a method for relieving dry skin and reducing the appearance of dry skin comprises providing an enzyme source that contains a protease enzyme. An extract of the enzyme source is produced and the protease enzyme is derived from the enzyme source extract. The protease enzyme is introduced into a composition, for example, a skin moisturizing composition. The composition is applied to dry skin and relieves the symptoms of dry skin and reduces the appearance of dry skin. This method is particularly effective in reducing the appearance of dry skin in individuals with darker-toned skin and/or darker skin pigmentation.

A further embodiment of the invention provides a method for digesting corneodesmosomal linkages between corneocyte cells. The method comprises providing an enzyme source that contains a protease enzyme. An extract of the enzyme source is produced and the protease enzyme is derived from the enzyme source extract. The protease enzyme is applied to a plurality of corneocyte cells that are connected by corneodesmosomal linkages, and the protease enzyme digests the corneodesmosomal linkages.

By way of definition, where a phrase similar to the phrase “at least one of A, B, and C” is used in the claims and/or elsewhere in the specification, it is intended that the phrase be interpreted to mean that A alone may be present in an embodiment, B alone may be present in an embodiment, or C alone may be present in an embodiment, or that any combination of the elements A, B and C may be present in a single embodiment; for example, A and B, A and C, B and C, or A and B and C may be present in a single embodiment.

Various principles of the invention have been described in exemplary embodiments. However, many combinations and modifications of the above-described structures, arrangements, proportions, elements, materials, and components, used in the practice of the invention, in addition to those not specifically described, can be varied without departing from those principles.

1. A skin care moisturizer, comprising:
a continuous phase, comprising:
a liquid, wherein the liquid comprises water;
an extract from at least one of a papaya and a pineapple, comprising a protease enzyme; and
at least one conditioning agent, wherein the at least one conditioning agent is water soluble; and
a dispersed phase, comprising a component that is not miscible with water.

2. A skin care moisturizer according to claim 1, wherein the continuous phase further comprises at least one of a humectant, a conditioning polymer, a color, a pH adjuster, a stabilizer, a preservative, and a vitamin.

3. A skin care moisturizer according to claim 1, wherein the dispersed phase further comprises at least one of an oil, a fatty acid, a lipid, and an oil-soluble vitamin.

4. A composition for skin care, comprising:
a first phase;
a second phase; and
an extract comprising an enzyme.

5. A composition according to claim 4, wherein the first phase is a continuous phase and the second phase is a dispersed phase.

6. A composition according to claim 4, wherein the first phase is a dispersed phase and the second phase is a continuous phase.

7. A composition according to claim 4, wherein the extract comprises a papaya extract.

8. A composition according to claim 4, wherein the extract comprises a pineapple extract.

9. A composition according to claim 4, wherein the first phase comprises a water-soluble skin conditioning agent.

10. A composition according to claim 4, wherein the first phase comprises an adjuvant ingredient.

11. A composition according to claim 10, wherein the adjuvant ingredient comprises at least one of a color, a fragrance, a pH adjuster, a stabilizer, a preservative, and a vitamin.

12. A composition according to claim 4, wherein the second phase comprises a skin conditioning agent.

13. A composition according to claim 12, wherein the skin conditioning agent is not miscible with water.

14. A composition according to claim 4, wherein the second phase comprises at least one of an oil, a fatty acid, a lipid, and an oil-soluble vitamin.

15. A method for treating dry skin, comprising the steps of:
providing an enzyme source and a base composition;
extracting a protease enzyme from the enzyme source;
introducing the protease enzyme into the base composition, forming a skin moisturizer; and
applying the skin moisturizer to the dry skin, thereby treating the dry skin.

16. A method according to claim 15, wherein the enzyme source comprises at least one of a papaya extract and a pineapple extract.

17. A method for treating dry skin, comprising the steps of:
providing an extract and a treatment area, wherein the treatment area comprises a plurality of corneocytes;
applying the extract to the treatment area; and
removing at least one of the plurality of corneocytes, thereby treating the dry skin.

18. A method according to claim 17, wherein the extract comprises a protease enzyme.

19. A method according to claim 17, wherein the treatment area further comprises a plurality of corneodesmosomal linkages.

20. A method according to claim 19, further comprising the step of digesting at least one of the plurality of corneodesmosomal linkages.

21. A method according to claim 17, wherein the step of applying the extract to the treatment area comprises the step of continuing to apply the extract to the treatment area for a period of twenty-one (21) days.

22. A method according to claim 17, wherein the extract comprises at least one of a pineapple juice extract and a papaya juice extract.
